(19) United States
(12) Patent Application Publication
(10) Pub. No.: US 2017/0119957 A1
(43) Pub. Date: May 4, 2017

(54) PORTABLE INTRAVENOUS BAG POSITIVE PRESSURE DEVICE

(71) Applicants: I-SHUO CHAO, Taichung (TW); CHIH-HUNG LIN, Taoyuan (TW)

(72) Inventor: I-SHUO CHAO, TAICHUNG (TW)

(73) Assignee: CHIH-HUNG LIN, Taoyuan (TW)

(21) Appl. No.: 15/290,112

(22) Filed: Oct. 11, 2016

(30) Foreign Application Priority Data
Oct. 30, 2015 (TW) 104217459

(51) Int. Cl. A61M 5/14; A61J 1/10; A61M 5/142

(52) U.S. Cl. CPC A61M 5/1415; A61M 5/14244; A61J 1/10; A61M 2005/1416

(57) ABSTRACT

A portable intravenous bag positive pressure device includes a main body and an elastic positioning unit. The main body is to encircle the arm of a patient at an appropriate position with its adjustable positioning belt to secure the main body. An intravenous bag is placed into an accommodation space formed between a positioning seat of the main body and the elastic positioning unit. The elastic positioning unit applies positive pressure to the intravenous bag to continuously output an infusion liquid for infusion therapy. The device has a simple structure and is convenient for wearing and able to secure the intravenous bag firmly on the arm of the patient. It is beneficial for the patient to carry the intravenous bag and move around during infusion therapy.

### FIELD OF THE INVENTION

The present invention relates to a portable intravenous bag positive pressure device which has a simple structure and is convenient for wearing and able to secure an intravenous bag firmly on the arm of a patient. It is beneficial for the patient to carry the intravenous bag and move around during infusion therapy.

### BACKGROUND OF THE INVENTION

When a patient needs to have an intravenous drip for some reasons, the intravenous bag is hanged on an infusion support to keep it higher than the injection position where the needle is inserted in the human body to ensure that the liquid in the intravenous bag (bottle) will have sufficient pressure to infuse the liquid into the human blood.

In general, when the patient having an intravenous drip wants to leave the hospital bed or ward, the intravenous bag (bottle) is hanged on a movable infusion support or an infusion pole provided on a wheelchair, such that the patent can have the infusion therapy continuously.

Although the infusion support can be used for the patient to have infusion therapy continuously when it is necessary to leave the hospital bed or ward, it is inconvenient for use. A conventional infusion support is a long frame. The upper end of the infusion support is provided with a hook for hanging the intravenous bag (bottle). For the patient to move around, the lower end of the infusion support is provided with casters. It is inconvenient and slow to push the frame forward. In case the space is not big enough, such as in a toilet, it is not easy to carry the infusion support into the toilet. If the patient goes to the toilet with one hand to take the intravenous bag (bottle) along, it is very troublesome and inconvenient. In addition, if the patient having an intravenous drip wants to leave the ward to take a walk outdoors and takes the intravenous bag (bottle) along, his/her arm will be sore after a period of time.

Therefore, how to develop a device which has a simple structure and is convenient for wearing and can secure an intravenous bag firmly is the applicant's goal.

Accordingly, the inventor of the present invention has devoted himself based on his many years of practical experiences to solve this problem.

### SUMMARY OF THE INVENTION

The primary object of the present invention is to improve the shortcomings of the prior art and to provide a portable intravenous bag positive pressure device. The device has a simple structure and is convenient for wearing and able to secure an intravenous bag firmly on the arm of a patient to output an infusion liquid continuously. It is beneficial for the patient to carry the intravenous bag conveniently and move around during infusion therapy.

In order to achieve the aforesaid object, the present invention is to provide a portable intravenous bag positive pressure device wound on the arm when in use. The portable intravenous bag positive pressure device comprises a main body and an elastic positioning unit. The main body has a positioning seat, an adjustable positioning belt and a positioning portion respectively extending from two sides of the positioning seat. The elastic positioning unit is an elastic member coupled to an outer side of the positioning seat to form an accommodation space between the elastic positioning unit and the positioning seat for receiving an intravenous bag therein. The main body is to encircle the arm of the patient by using the adjustable positioning belt to insert through the positioning portion to be secured at an appropriate position. After that, the intravenous bag is placed into the accommodation space formed between the positioning seat of the main body and the elastic positioning unit. The elastic positioning unit is an elastic member to apply positive pressure to the intravenous bag accommodated in the accommodation space to continuously output the infusion liquid for infusion therapy.

### BRIEF DESCRIPTION OF THE DRAWINGS

FIG. 1 is a perspective view in accordance with a first embodiment of the present invention;

FIG. 2 is a schematic view of FIG. 1 when in use;

FIG. 3 is a partial sectional view of FIG. 1;

FIG. 4 is a schematic view of the first embodiment of the present invention when in use;

FIG. 5 is a schematic view of another embodiment of FIG. 1;

FIG. 6 is a schematic view of FIG. 5 when in use;

FIG. 7 is a perspective view in accordance with a second embodiment of the present invention;

FIG. 8 is a schematic view of FIG. 7 when in use;

FIG. 9 is a partial sectional view of FIG. 7;

FIG. 10 is a schematic view of the second embodiment of the present invention when in use;

FIG. 11 is a schematic view of another embodiment of FIG. 7; and

FIG. 12 is a schematic view of FIG. 11 when in use.

### DESCRIPTION OF THE PREFERRED EMBODIMENTS

Embodiments of the present invention will now be described, by way of example only, with reference to the accompanying drawings.

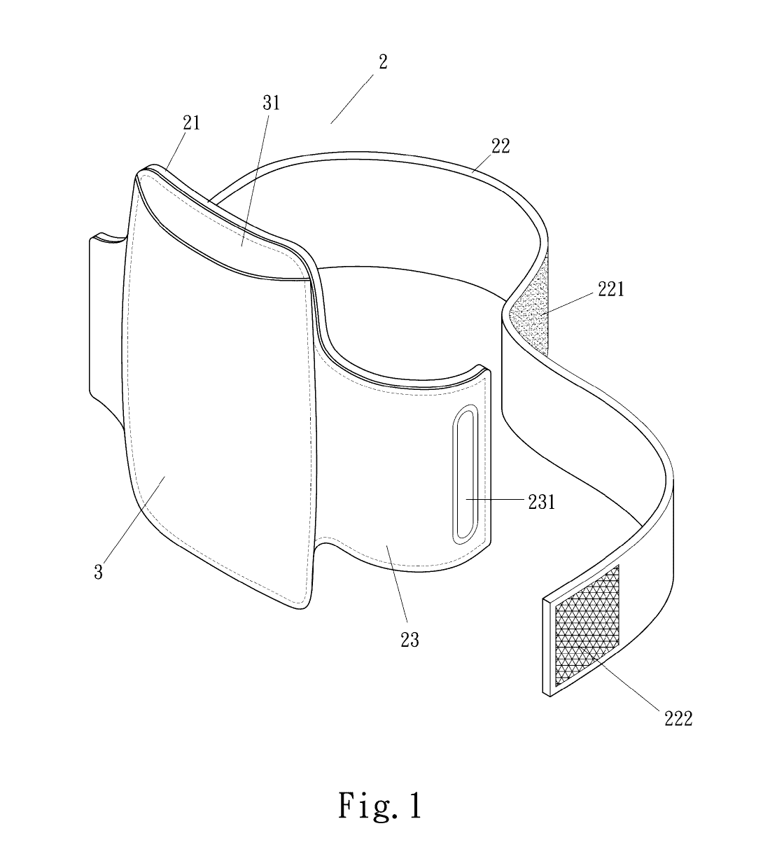

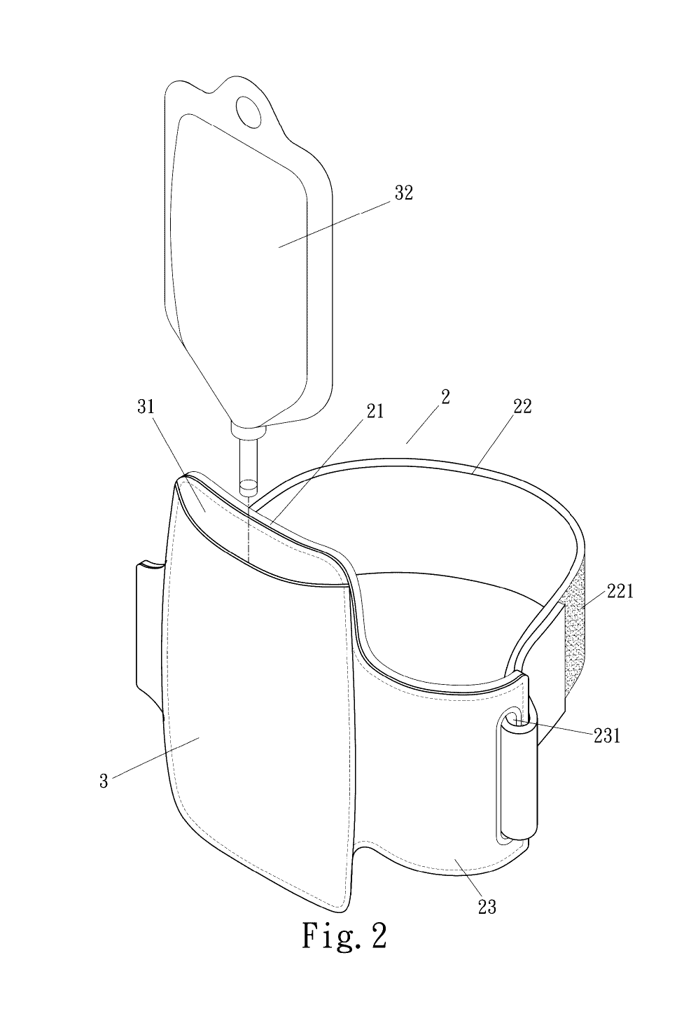

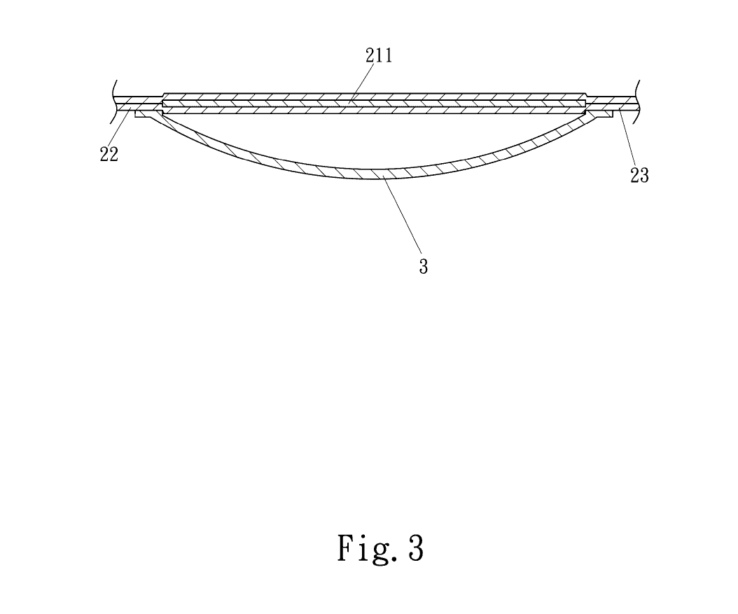

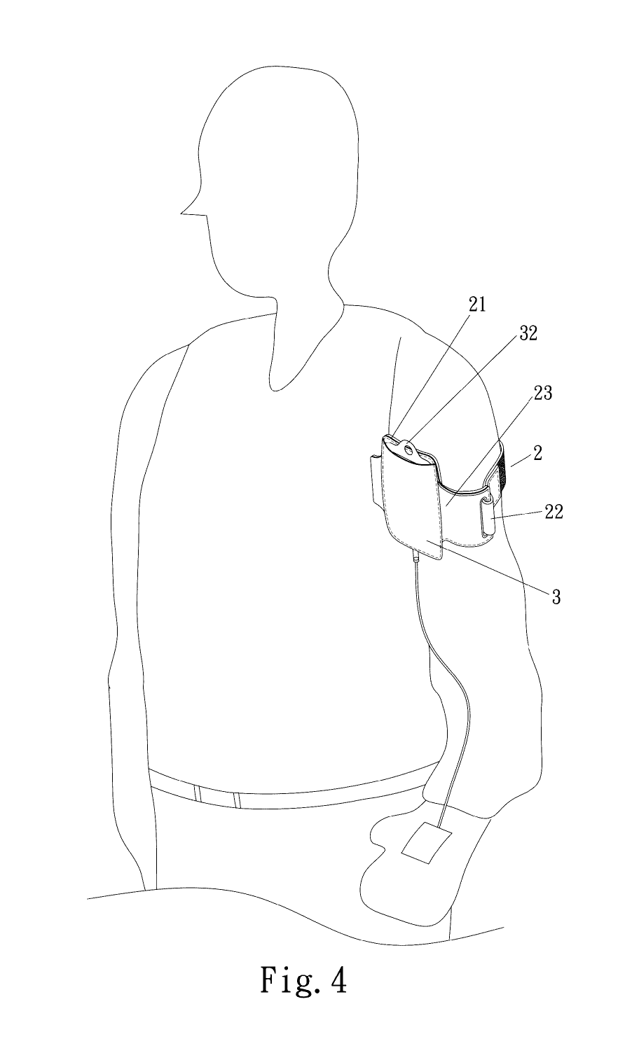

Referring to FIG. 1 to FIG. 4, a portable intravenous bag positive pressure device in accordance with a first embodiment of the present invention comprises a main body 2 and an elastic positioning unit 3.

The main body 2 has a positioning seat 21, an adjustable positioning belt 22 and a positioning portion 23 respectively extending from two sides of the positioning seat 21. The positioning seat 21 is provided with a positioning board 211 inside the positioning seat 21. The positioning board 211 is hard, not easy to cause deformation. The adjustable positioning belt 22 is provided with a first coupling member 221 and a second coupling member 222. The first coupling member 221 and the second coupling member 222 may be Velcro tapes or male and female buckles. The first coupling member 221 and the second coupling member 222 are spaced a determined distance apart from each other. The positioning portion 23 has a slot 231. When in use, the free end of the adjustable positioning belt 22 is inserted through the slot 231 and the first coupling member 221 is coupled to the second coupling member 222, such that the positioning seat 21, the adjustable positioning belt 22, and the positioning portion 23 are formed in a circle.

The elastic positioning unit 3 is an elastic member coupled to the outer side of the positioning seat 21 of the main body 2 to form an accommodation space 31 between the elastic positioning unit 3 and the positioning seat 21. The accommodation space 31 is configured to receive an intravenous bag 32 filled with an infusion liquid, such that the intravenous bag 32 is confined within the accommodation space 31.

When a patient having an intravenous drip needs to walk and leave the hospital bed or ward for some reasons, he/she may use the portable intravenous bag positive pressure device of the present invention to continue the intravenous drip. The intravenous bag 32 is first placed into the accommodation space 31 formed between the elastic positioning unit 3 and the outer side of the positioning seat 21 of the main body 2. Through the adjustable positioning belt 22 and the positioning portion 23 at the two sides of the positioning seat 21 of the main body 2, the free end of the adjustable positioning belt 22 is inserted through the slot 231 of the positioning portion 23, and the first coupling member 221 is coupled to the second coupling member 222, such that the positioning seat 21, the adjustable positioning belt 22, and the positioning portion 23 are formed in a circle to encircle the arm of the patient. In addition, the positioning seat 21 of the main body 2 has the positioning board 211 which is hard, not easy to cause deformation. When the intravenous bag 32 is placed into the accommodation space 31 formed between the elastic positioning unit 3 and the outer side of the positioning seat 21 of the main body 2, the entire intravenous bag 32 is only biased by the elastic positioning unit 3 to apply a one-way positive pressure force to the intravenous bag 32 to continuously output the infusion liquid for infusion therapy. The device has a simple structure and is convenient for wearing, so that the intravenous bag can be firmly placed on the arm of the patient. It is beneficial for the patient to carry the intravenous bag and move around during infusion therapy.

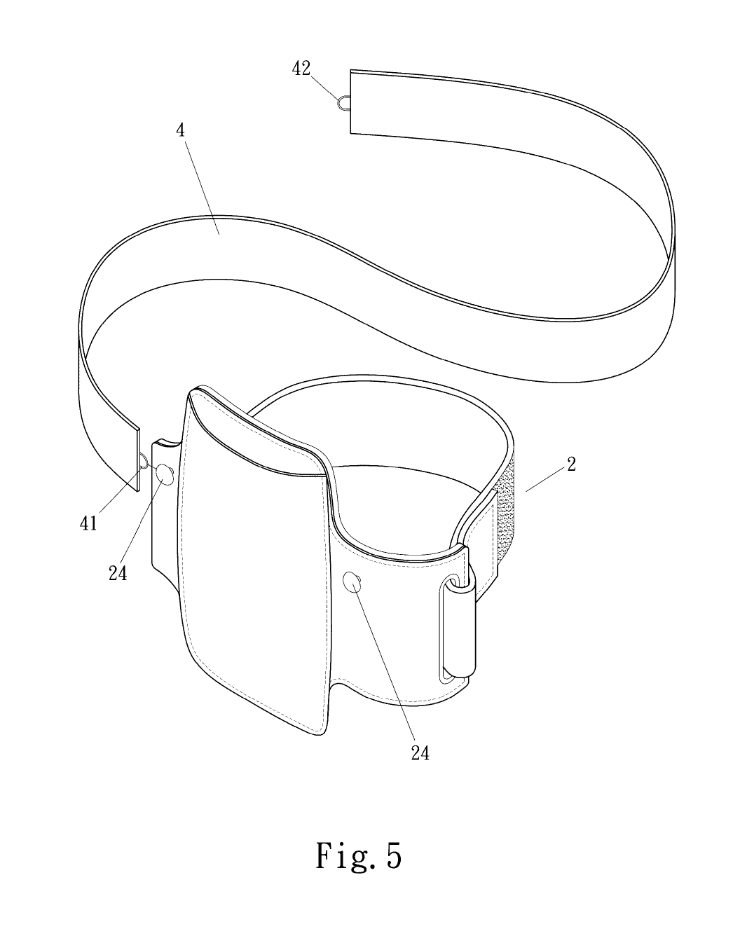

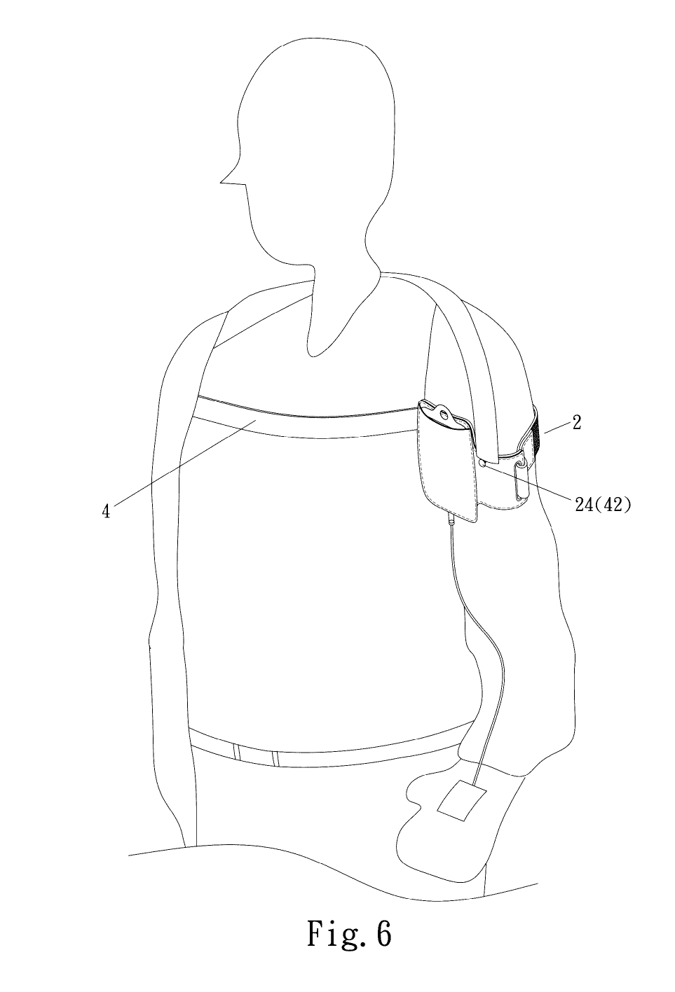

Referring to FIG. 5 and FIG. 6, the portable intravenous bag positive pressure device further comprises a strip 4 with a suitable length. The main body 2 is provided with a connecting member 24. One end 41 of the strip 4 is first connected to the main body 2, and another end 42 goes around the armpit and the neck of the patient and then is connected to another connecting member 24 of the main body 2. The portable intravenous bag positive pressure device can be secured on the arm of the patient more firmly.

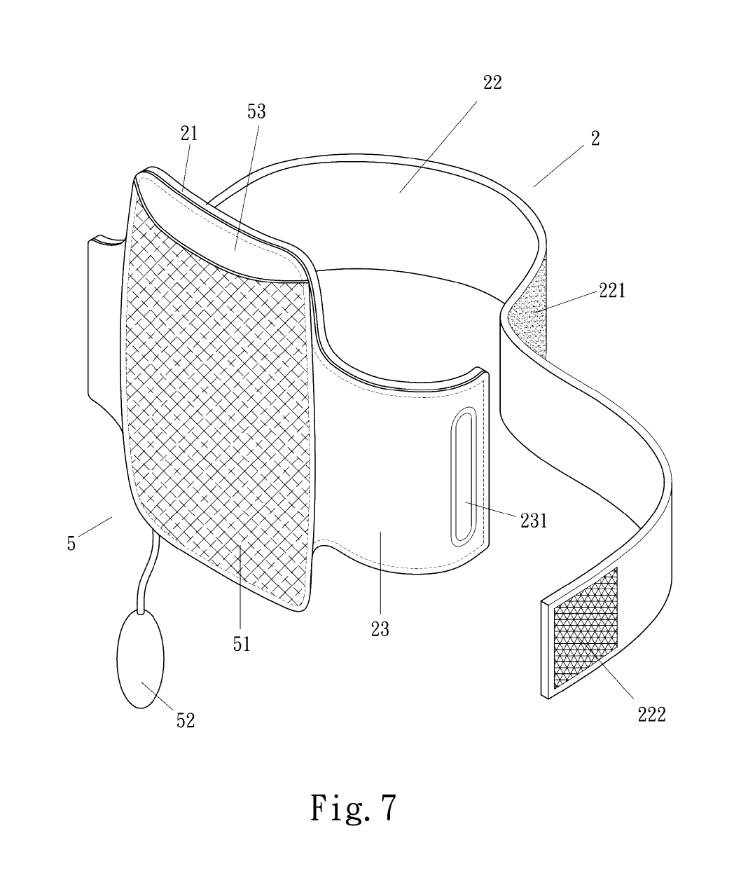

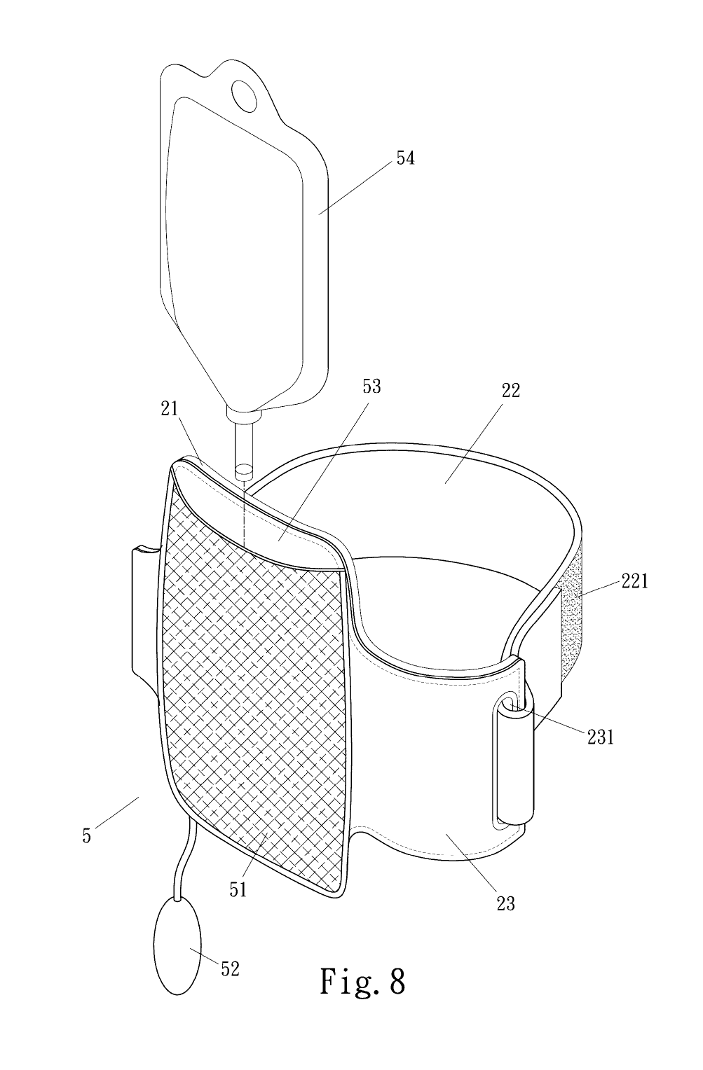

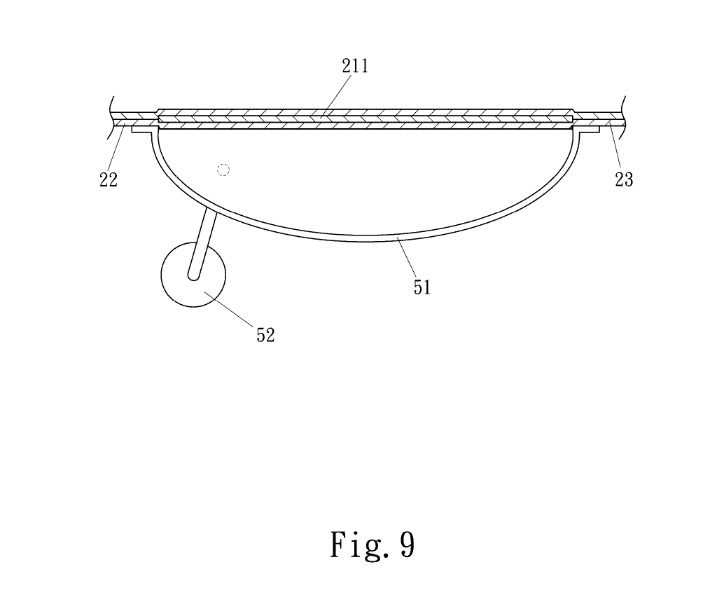

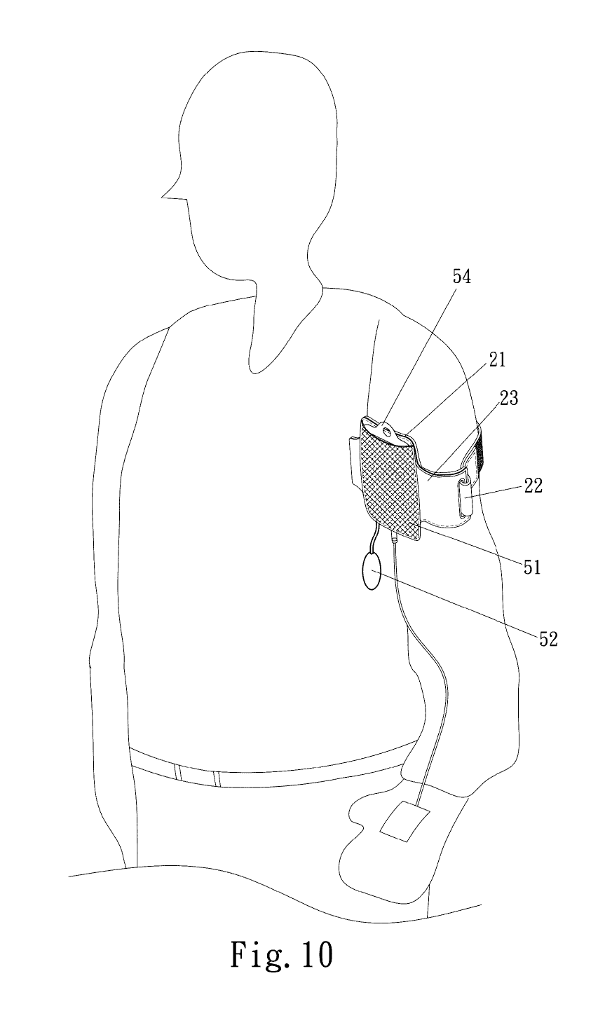

Referring to FIG. 7 to FIG. 10, a portable intravenous bag positive pressure device in accordance with a second embodiment of the present invention comprises a main body 2 and an elastic positioning unit 5.

The main body 2 has a positioning seat 21, an adjustable positioning belt 22 and a positioning portion 23 respectively extending from two sides of the positioning seat 21. The positioning seat 21 is provided with a positioning board 211 inside the positioning seat 21. The positioning board 211 is hard, not easy to cause deformation. The adjustable positioning belt 22 is provided with a first coupling member 221 and a second coupling member 222. The first coupling member 221 and the second coupling member 222 may be Velcro tapes or male and female buckles. The first coupling member 221 and the second coupling member 222 are spaced a determined distance apart from each other. The positioning portion 23 has a slot 231. When in use, the free end of the adjustable positioning belt 22 is inserted through the slot 231 and the first coupling member 221 is coupled to the second coupling member 222, such that the positioning seat 21, the adjustable positioning belt 22, and the positioning portion 23 are formed in a circle.

The elastic positioning unit 5 comprises an air storage bag 51 and a manual inflation device 52 for inflating the air storage bag 51. The interior of the air storage bag 51 is hollow. The air storage bag 51 is coupled to the outer side of the positioning seat 21 of the main body 2 to form an accommodation space 53 between the elastic positioning unit 5 and the positioning seat 21. The accommodation space 53 is configured to receive an intravenous bag 54 filled with an infusion liquid.

When a patient having an intravenous drip needs to walk and leave the hospital bed or ward for any reason, he/she may use the portable intravenous bag positive pressure device of the present invention to continue the intravenous drip. The intravenous bag 54 is first placed into the accommodation space 53 formed between the elastic positioning unit 5 and the outer side of the positioning seat 21 of the main body 2. Through the adjustable positioning belt 22 and the positioning portion 23 at the two sides of the positioning seat 21 of the main body 2, the free end of the adjustable positioning belt 22 is inserted through the slot 231 of the positioning portion 23, and the first coupling member 221 is coupled to the second coupling member 222, such that the positioning seat 21, the adjustable positioning belt 22, and the positioning portion 23 are formed in a circle to encircle the arm of the patient. In addition, the positioning seat 21 of the main body 2 has the positioning board 211 which is hard, not easy to cause deformation. When the intravenous bag 54 is placed into the accommodation space 53 formed between the elastic positioning unit 5 and the outer side of the positioning seat 21 of the main body 2, the air storage bag 51 is filled with gas through the manual inflation device 52 of the elastic positioning unit 5 so that the elastic positioning unit 51 filled with gas applies a one-way positive pressure force to the entire intravenous bag 54 to continuously output the infusion liquid for infusion therapy. The device has a simple structure and is convenient for wearing, so that the intravenous bag can be firmly placed on the arm of the patient. It is beneficial for the patient to carry the intravenous bag and move around during infusion therapy.

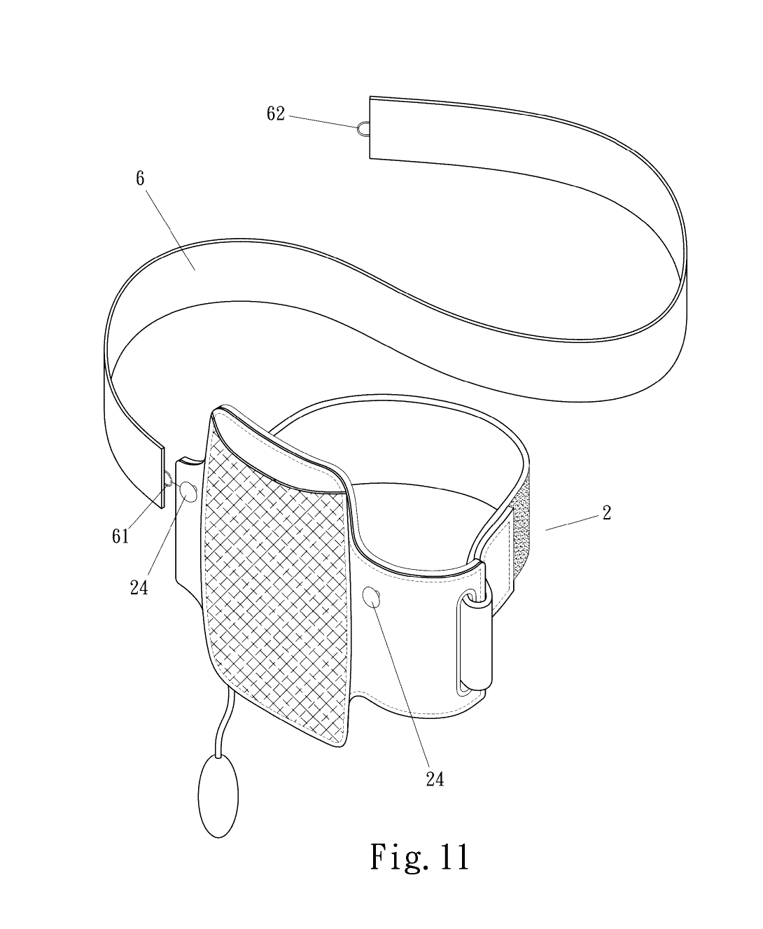

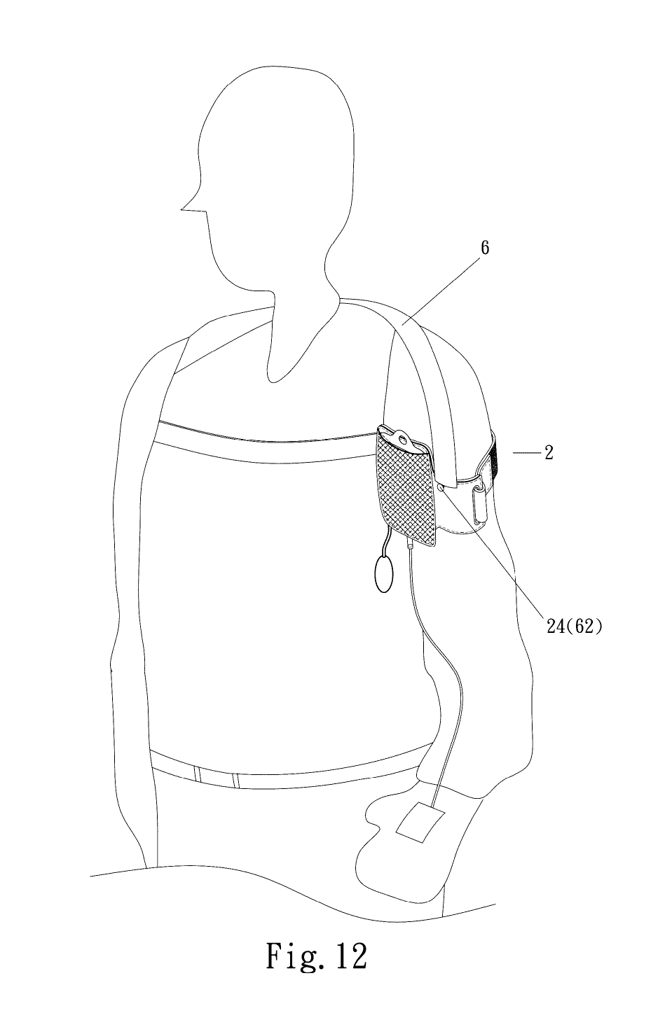

Referring to FIG. 11 and FIG. 12, the portable intravenous bag positive pressure device further comprises a strip 6 with a suitable length. The main body 2 is provided with a connecting member 24. One end 61 of the strip 6 is first connected to the main body 2, and another end 62 goes around the armpit and the neck of the patient and then is connected to another connecting member 24 of the main body 2. The portable intravenous bag positive pressure device can be secured on the arm of the patient more firmly.

Thereby, the present invention is to provide a portable structure for an intravenous bag. The main body is to encircle the arm of the patient by using the adjustable positioning belt to insert through the positioning portion to be secured at an appropriate position. After that, the intravenous bag is placed into the accommodation space formed between the positioning seat of the main body and the elastic positioning unit. The elastic positioning unit is an elastic member to apply positive pressure to the intravenous bag accommodated in the accommodation space to continuously output the infusion liquid for infusion therapy. The device has a simple structure and is convenient for wearing, so that the intravenous bag can be firmly placed on the arm of the patient. It is beneficial for the patient to carry the intravenous bag and move around during infusion therapy.

The present invention is to solve the shortcomings of the prior art. Its effects and advantages are obvious. The present invention has never been seen and disclosed in any publication, so it meets the requirements of patentability.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A portable intravenous bag positive pressure device, comprising:
a main body, having a positioning seat, an adjustable positioning belt and a positioning portion respectively extending from two sides of the positioning seat; wherein the adjustable positioning belt is provided with a first coupling member and a second coupling member, the first coupling member and the second coupling member are spaced a determined distance apart from each other; wherein the positioning portion has a slot, when in use, a free end of the adjustable positioning belt is inserted through the slot and the first coupling member is coupled to the second coupling member, such that the positioning seat, the adjustable positioning belt, and the positioning portion are formed in a circle; and
an elastic positioning unit, being an elastic member coupled to an outer side of the positioning seat of the main body to form an accommodation space between the elastic positioning unit and the positioning seat of the main body, the accommodation space being configured to receive an intravenous bag filled with an infusion liquid for the intravenous bag to be confined within the accommodation space.

2. The portable intravenous bag positive pressure device as claimed in claim 1, wherein the positioning seat of the main body is provided with a positioning board inside the positioning seat, and the positioning board is hard, not easy to cause deformation.

3. The portable intravenous bag positive pressure device as claimed in claim 1, wherein the first coupling member and the second coupling member are Velcro tapes.

4. The portable intravenous bag positive pressure device as claimed in claim 1, wherein the first coupling member and the second coupling member are male and female buckles.

5. The portable intravenous bag positive pressure device as claimed in claim 1, further comprising a strip, the main body being provided with connecting members, two ends of the strip being connected to the connecting members of the main body, respectively.

6. A portable intravenous bag positive pressure device, comprising:
a main body, having a positioning seat, an adjustable positioning belt and a positioning portion respectively extending from two sides of the positioning seat; wherein the adjustable positioning belt is provided with a first coupling member and a second coupling member, the first coupling member and the second coupling member are spaced a determined distance apart from each other; wherein the positioning portion has a slot, when in use, a free end of the adjustable positioning belt is inserted through the slot and the first coupling member is coupled to the second coupling member, such that the positioning seat, the adjustable positioning belt, and the positioning portion are formed in a circle; and
an elastic positioning unit, comprising an air storage bag and a manual inflation device for inflating the air storage bag, an interior of the air storage bag being hollow, the air storage bag being coupled to an outer side of the positioning seat of the main body to form an accommodation space between the elastic positioning unit and the positioning seat, the accommodation space being configured to receive an intravenous bag filled with an infusion liquid.

7. The portable intravenous bag positive pressure device as claimed in claim 6, wherein the positioning seat of the main body is provided with a positioning board inside the positioning seat, and the positioning board is hard, not easy to cause deformation.

8. The portable intravenous bag positive pressure device as claimed in claim 6, wherein the first coupling member and the second coupling member are Velcro tapes.

9. The portable intravenous bag positive pressure device as claimed in claim 6, wherein the first coupling member and the second coupling member are male and female buckles.

10. The portable intravenous bag positive pressure device as claimed in claim 6, further comprising a strip, the main body being provided with connecting members, two ends of the strip being connected to the connecting members of the main body, respectively.
